(19) United States
(12) Patent Application Publication
(10) Pub. No.: US 2009/0270495 A1
(43) Pub. Date: Oct. 29, 2009

(54) Inhibitors of Phosphodiesterase Types 1 To 5 Based on Dioclein, Floranol, and Analogs Thereof

(76) Inventors: Virginia Soares Lemos, Belo Horizonte-MG (BR); Stayner De Franca Cortes, Belo Horizonte-MG (BR); Bruno Almeida Resende, Belo Horizonte-MG (BR); Roberta Lins Goncalves, Belo Horizonte-MG (BR); Ruben Dario Sinisterra Milan, Ouro Preto-MG (BR); Martine Schmitt, Strasbourg (FR); Claire Lugnier, Strasbourg (FR); Jean-Jacques Bourguignon, Illkirch (FR)

Correspondence Address: NIXON & VANDERHYE, PC, 901 NORTH GLEBE ROAD, 11TH FLOOR, ARLINGTON, VA, 22203, US

(21) Appl. No.: 11/887,564

(22) PCT Filed: Mar. 30, 2006
(86) PCT No.: PCT/BR2006/000060

§ 371 Date: Mar. 24, 2008

(30) Foreign Application Priority Data
Mar. 31, 2005 (BR) PI0502411-0

(51) Int. Cl. A61K 31/352; A61P 9/10

(52) U.S. Cl. 514/456

(57) ABSTRACT

We disclose substances and a process of developing such substances as potent and selective inhibitors of isoforms of phosphodiesterases of types 1 to 5 (PDE1, PDE2, PDE3, PDE4, PDE5) based on two flavonoids: dioclein, floranol and natural or synthetic analogs thereof. They may be associated with cyclodextrins in inclusion complexes or using a biodegradable or non-biodegradable polymer, such as PLGA, PLA, PGA or mixtures thereof in controlled release devices. Their respective pharmaceutical compositions as well as pharmaceutical and pharmacologically acceptable excipients may be used for the study and treatment of cardiovascular diseases and associated products.

The present invention relates to a process of developing substances as potent and selective inhibitors of the isoforms of phosphodiesterases of types 1 to 5 (PDE1, PDE2, PDE3, PDE4, PDE5) based on dioclein, floranol or natural or synthetic analogs; associated to inclusion compounds with the cyclodextrins and to the controlled-release devices using biodegradable or non-biodegradable polymers, such as PLGA, PLA, PGA or mixtures thereof; their respective pharmaceutical compositions for the study and treatment of cardiovascular diseases and associated products.

The substances developed in the present invention have been tested for their ability of inhibiting different isoforms of PDEs. These are also the first substances, and their pharmaceutical compositions, in the therapeutical arsenal capable of inhibiting, in a potent and selective manner, the isoform of type-1 (PDE1) phosphodiesterase.

The present invention employs two flavonoids as forms that inhibit phosphodiesterases of types 1 to 5: dioclein and floranol, as well as their analogs and pharmaceutical compositions, using the cyclodextrins and their inclusion compounds, as well as pharmaceutical and pharmacologically acceptable excipients.

Phosphodiesterases are non-specific enzymes that catalyze the degradation of cyclic nucleotides AMPc (cyclic adenosine monophosphate) and GMPc (cyclic guanosine monophosphate), which act on several organs of the human body and of other mammals.

AMPc and GMPc are second messengers that play a key role in regulating numberless cellular functions such as metabolism, contractility, motility and transcription in practically all the types of cells, including those of the cardiovascular system. PDEs represent the only way to degradation of AMPc and GMPc and, therefore, are important regulators of the cellular function [Polson J. B. and Strada S. J., Ann. Rev. Pharmacol. Toxicol., (1996) 36, 403-427].

The AMPC is a nucleotide produced from ATP in response to hormonal stimulation of receptors of the cell surface. It is an important molecule in the transmission of intracellular signals. It acts as a signaling molecule, activating the protein kinase A and, when hydrolyzed, generates AMP by a phosphodiesterase. Once formed, the AMPc causes intracellular effects, thus being considered an intercellular hormonal mediator.

The GMPc is a nucleotide produced from GTP by a guanylate cyclase. The guanylate cyclase can be activated in several ways, one of them being by nitric oxide, which is spreads from the endothelium to the smooth muscle cells of the vessels. The atrial natriuretic peptide also stimulates the formation of GMPc. The GMPc activates the protein kinase G, which in turn, can act in the smooth muscle to stimulate the opening of potassium channels, causing hyperpolarization of the cell. It can also act by activating the pump Ca²⁺/K⁺-ATPase, which causes calcium to come out of the cytoplasm to the extra cellular medium and from the cytoplasm into said sarcoplasmic reticulum. This causes a decrease of the intracellular free calcium. Further, the protein kinase G phosphorilates the contractile fibers, making them less sensible to calcium. These effects make the GMPc a messenger molecule that reduces the muscular contraction that is clearly dependent upon calcium. The concentration of GMPc is important in numberless physiologic events, as in the change of vascular tonus, erection and cellular proliferation. The phosphodiesterases act to reduce the life-span of the GMPc.

Until now 11 different families of PDEs isoenzymes have been described and knowing the exact physiological role that each one of them plays is still complex and under study [Soderlining S. H. and Beavo J. A., Curr. Opin. Cell Biol., (2000) 12, 174-179]. In each family there are multiple isoforms as a result of the existence of multiple genes and alternative “splicing”. The various phosphodiesterases existing differ in their primary structure, ability of hydrolyzing AMPc and GMPC, tissular and intracellular distribution and sensitivity to pharmacological modulators and inhibitors [O'Donnel J. M. and Zhang H. T., Trends Pharmacol., Sci., (2004) 25, 158-163].

The PDEs 1 are present in the cardiovascular system (vessels and cardiomyocytes), in the brain and in other nerve tissues, and also in the kidneys and in the adrenal medulla. They are activated by Ca⁺² and calmodulin (CaM). The variants PDE1A and PDE1B selectively hydrolyze GMPc, but the variant PDE1C hydrolyzes both AMPc and GMPc. The PDE1A have been implied in the tolerance developed by the vessels to organic nitrates and, therefore, selective inhibitors of this isoenzyme could be used as a therapeutic tool for limiting tolerance to nitrates. The PDE1C is implied in the proliferation of the vascular smooth muscular cells. The use of selective inhibitors for this latter isoform could minimize proliferative responses found in the injury and inflammation caused by the angioplasty, in atherosclerosis, in arterial hypertension, etc. The PDE1C has also been implied in the secretion of insulin. In the cardiovascular system, the PDE1 has also been implied in the control of the brain circulation (Maurice D. H. et al., Mol. Pharmacol, (2003) 64, 533-546].

The PDES 2 are stimulated by the GMPc and hydrolyze both AMPc and GMPc. They are found in the platelets, in the cardiomyocytes, endothelial and vascular cells, and adrenal granular cells. The natriuretic peptides and donors of nitric oxide increase the cellular GMPc and activate the PDE2 in some of these cells [Maurice D. H et al., Mol. Pharmacol., (1003) 64,533-546].

The PDEs 3 are present in the blood vessels, heart, megakaryocytes, oocytes, liver, adipocytes, brain, renal collecting ducts and developing sperm. They hydrolyze both AMPc and GMPc. They are activated by the protein kinase A and by the protein kinase B or an insulin-activating kinase and are inhibited by the GMPc. At the cellular level, the PDEs 3 play an important role as regulators of the effects of insulin on the metabolism of lipids and carbohydrates, act in controlling the activity of the L-type Ca²⁺ channels in the cardiomyocytes, are implied in the process of controlling the tonus and vascular proliferation and in inflammatory processes [Maurice D. H. et al., Mol. Pharmacol, (2003) 64, 533-546].

The PDEs 4 are found in almost all the types of cells, except in the platelets. They are characterized by hydrolyzing specifically AMPc. This family of PDEs consists of 4 types of independently encoded enzymes (PDE4A-PDE4D). At the molecular level, they act to raise the levels of AMPc. The PDE4 are widely implied in immunological and inflammatory disorders, as well as in the depression physiopathology [Maurice D. H. et al., Mol. Pharmacol, (2003) 64, 533-546].

The PDEs 5 hydrolyze specifically GMPc. This family consists of a single gene, which encodes 3 different proteins (PDE5A1-3). The PDE5 is present in numberless tissues, like the brain, lung, platelets, visceral and vascular smooth muscle and kidneys. In inhibitors of PDE5, like sildenafil (Viagra®), are used in erectile dysfunction and in pulmonary hypertension [Lin C. S. et al., Urology, (2003) 61, 685-692].

It is known that many pathologies related with the mechanism of functioning of the phosphodiesterases are being studied and the inhibition of the known isoforms has been a treatment mechanism for various diseases. Thus, the PDE1-PDE5 inhibitors have been used for the treatment of the erectile-dysfunction problems [Rosen R. C and Kostis J. B., Am. J. Cardiol., (2003) 92, 9M-18M]; in the treatment of asthma and other inflammatory diseases [Torphy T. J., Am. J. Respir. Crit. Care, Med., (1997) 157, 351-370].

Flavonoids are compounds existing since billions of years and can be found in a wide variety of plants. They are responsible for the colorful aspect of leaves and flowers, and may also be present in other parts of plants. There are six classes of flavonoids: flavanones, flavones, flavanes, flavonols, isoflavonoids, anthocyanines, which vary in their structural characteristics around the heterocyclic oxygen ring. The differences lie in the absorption of each class [Peterson J. and Dwyer J., Nutr. Res., (1998) 18, 1995-2018].

This broad class of substances of natural origin, the synthesis of which does not occur in the human species, has important pharmacological properties, which act on biological systems. Consequently, many of these properties act in a beneficial way on human health.

There are over 4,000 different flavonoids, which exhibit various biochemical and pharmacological activities, such as anti-oxidant anti-inflammatory, anti-allergic, antiviral and anticarcinogenic action. In plants, beside the biochemical activities, the flavonoids act as precursors of toxic substances, pigments and light protectors.

Among the several pharmacological activities attributed to the flavonoids, one points out the anti-oxidant capacity, anti-inflammatory activities and vasodilating effect; anti-allergic action; activity against the development of tumors, antihepatotoxic, antiulcerogenic; anti-platelet, as well as antimicrobial and antiviral actions. It is also known that the flavonoids can inhibit various stages of the processes that are directly related with the beginning of atherosclerosis, like the activation of leucocytes, adhesion, aggregation and secretion of platelets [Hladovec J., Physiol. Bohemoslov. (1986) 35, 97-103], besides having hypolipidemic activities [Matsuda et al., J. Ethonopharmacol. (1986) 17, 213-24] and increasing the activity of LDL receptors [Kirk et al., J. Nutr. (1998) 128, 954-959; www.polymar.com.br/saude/s flavonoides.php].

Flavonoids have also been studied as inhibitors of the action of enzymes. The literature reports the inhibiting activity of flavonoids for several types of enzymes, as for example, cyclooxygenase, estrogen synthase, glutathione synthase, lipoxygenase, xanthine oxydase, and phosphodiesterases [Peterson J. and Dwyer J., Nutr. Res., (1998), 18, 1995-2018].

The use of dioclein, floranol and analogs as inhibitors of the isoforms of phosphodiesterases PDE1, PDE2, PDE3, PDE4 and PDE5, and as models for the development of new pharmaceuticals was not found in the prior art. Further, it was not found in the prior art the process for preparing inclusion compounds between dioclein and floranol with cyclodextrins for use in oral formulations, as inhibitors of phosphodiesterases in their isoforms 1, 2, 3, 4 and 5, as well as for the study and treatment of degenerative chronic diseases like atherosclerosis, hypertension and related cardiovascular diseases and use thereof as models for the development of new pharmaceuticals, as well as their pharmaceutical compositions.

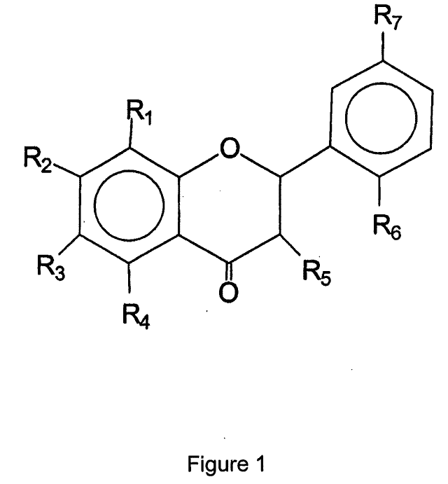

The present invention is characterized by the development of new substances with the chemical structure of formula 1, as potent and selective inhibitors of PDEs 1 to 5. It has also aims at the effect of compounds of FIG. 1, as preventives against cellular proliferation, vasodilator, anti-hypertensives, anti-inflammatories and as preventives against atherosclerosis.

In the formula of FIG. 1, R¹, R², R³,R⁴, R⁵, R⁶ and R⁷ are functional groups that may be the same or different and include, but are not limited to, hydrogen, hydroxyl, methoxyl and prenyl.

Dioclein (5, 2′,5′-trihydroxy-6,7-dimethoxyflavanone), FIG. 1, is a flavonoid of the class of the flavanones, a group of compounds found at high concentrations in citric fruits. The flavanones stand out for their bioactivity against certain types of cancer, especially colon cancer and breast cancer, and improve the venous and arterial circulation thanks to their platelet anti-aggregating, vasodilating properties, as well as inhibiting cellular adhesion at the plasmatic level. In addition, they exhibit analgesic, anti-allergic and anti-inflammatory properties.

Dioclein has been obtained from its synthesis by using the method described by Spearing P. et al. [J. Nat. Prod., (1997) 60, 399-400]. This flavonoid was first described upon its isolation from the ethanolic extract from Dioclea grandiflora. This plant is known for its medicinal value and occurs in the northeast of Brazil, especially in the regions of the so-called “caatinga” (stunted sparse forest) and “cerrado” (patches with stunted vegetation) [Jenkins T. et al., Phytochemistry, (1999) 52, 723-730. The analgesic effect of dioclein is known [Batista J. S. et al., J. Ethnopharmacol. (1995) 45, 207-210], in addition to their vasodilating properties [Lemos V. S et al., Eur. J. Pharmacol., (1999) 386, 41-46]. In spite of its three hydroxyls, the aromatic rings and a hetorocycle one confer to it a non-polar nature, having low solubility in water and being soluble in DMSO and methanol.

Dioclein has a limitation in its use due to its hydrophobicity, instability and little or no activity when administered by oral route. So, the present invention proposes a solution to the prior art, using the formation of inclusion compounds with cyclodextrins and their derivatives, and the obtainment of active pharmaceutical compositions having high bioavailability when applied in oral form.

Floranol, the chemical formula of which is described in FIG. 1, is a flavonoid of the class of the flavonones and exhibits vasodilating activity [Rezende B. A. et al., Planta Med. (2004) 70, 465-467].

Other phosphodiesterase inhibitors for the isoforms 2 and 5 are known, but few are available on the market for several reasons, either the high cost of researches or undesired side effects.

Few PDE2 inhibitors are known. Erythro-9-(2-hydroxyl-3-nonyl) adenine, a potent enzyme adenosine deaminase inhibitor, inhibits the activation of PDE2 by GMPc. This substance was tested on various tissues, but its potential clinical use is still unknown.

The inhibition of phosphodiesterase 3 and 4 relaxes the smooth muscles of the bronchi and pulmonary arteries, and the immunomodulatory and anti-inflammatory action results from the inhibition of isoenzyme-4. Mediators of inflammation released by mastocytes, lymphocytes T, macrophages, eosinophils and epithelial cells may be inhibited by the PDE4.

The PDE3 inhibitors do not have utilization in the clinical practice due to the association with cardiovascular problems, mainly in arrhythmias. The PDE4 have also the great limitation due to their side effects, mainly nauseas and vomit—this is because the vomit center is out of the hemato-encephalic barrier and the action of which cannot be dissociated from the anti-inflammatory effects [www.asmabronquica.com.br/pierre/33teofilina.pdf].

The known PDE3 inhibitors are inotropics and vasodilating drugs such as: cilostamide, milrinone, amrinone, enoximone, imazodan, indolidan, cilostazol and olprinone. [Manganiello V. C. et al., Arch. Biochem. Biophys., (1995) 322, 1-13]. Olprinone has been clinically tested for the treatment of intramuscular gastric acidosis and systemic inflammation after cardiopulmonary “bypass”. Cilostazol has an anti-platelet, vasodilating and antithrombotic action. It has been tested clinically for the treatment of angioplastic restenosis. However, it is expensive and also has adverse reactions, like headache, diarrhea, palpitations, tachycardia, and the use thereof being inadequate for patients with any type of heart problem [http://www.ukmi-nhs.uk/NewMaterial/html/docs/Cilostazol. pdf].

The most widely-known PDE4 inhibitor is Rolipram, which exhibits serious side effects, and its use is being restricted [Manganiello V. C. et al., Arch. Biochem. Biophys., (1995) 322, 1-13]. There is also a new drug to inhibit phosphodiesterases of type 4, namely BAY 19-8004, used for lung diseases such as inflammation of the bronchi, asthma and chronic coronary obstruction; but it has presented significant side effects only with respect to this latter disease, and its side effects are little known [Grootendorst D. C et al., Pulm. Pharmacol. Ther. (2003) 16, 341-347].

Cilomilast and roflumilast, two of other PDE4 inhibitors, have been clinically tested for use against asthma, chronic obstructive pulmonary disease and allergic rhinitis.

The inhibitors best known on the market are those suitable to act on PDE5, which act mainly on erectile-dysfunction-related problems, namely, sildenafil, vardenafil and tadalafil, exisulind and CP461. All these medicaments still have disadvantages with regard to their use. In the comparative analysis, the two latter pharmaceuticals exhibit more efficacy when compared with sildenafil, however, the long-term effects of the reiterated use of vardenafil and of tadalafil are not known—a reason that leads sildenafil to be more widely used. [Gresser U. and Gleiter C. H., Eur. J. Med. Res., (2000) 27, 435-446]. However, sildenafil, active principle of Viagra® still exhibits side effects such as headache, indigestion with possibility of reflux and rubor, besides momentary visual blurring [Goldstein I. et al., N. Engl. J. Med., (1998) 338, 1397-1404]. Sildenafil is also used for the treatment of pulmonary hypertension. Exisulind and CP461 are being tested for the treatment of various type of cancer.

Other phosphodiesterase inhibitors, among them natural inhibitors, are known, but little used in clinic for several reasons, such as excess of side effects, little selectivity in inhibiting various isoforms, the need for high dosages, among others.

Paraverin, which is a non-specific PDEs inhibitor, is used in clinic as vasodilator, especially for erectile dysfunction. It is a very cheep and effective drug, but it has strong side effects. A single application may cause fibrosis of the cavernous bodies of the penis. In addition, the priapism, a persistent erection (more than 4 hours), often painful, which is not followed by sexual desire, is quite high. [http:/www.lincx.com.br/lincx/atualizacao/ artigos/disfuncao_sexual.html]. It is also used topically as vasodilator in surgeries of cardiac revascularization.

Teofilin acts to inhibit the PDE enzymes of the types 3, 4 and 5. It is a compound originally extracted from black-tea leaves. Inhibition of PDEs 3 and 4 increases the intracellular concentrations of AMPc, and the inhibition of PDE 5 increases the levels of GMPc in the bronchial smooth musculature and in the inflammatory cells. It is being used over 50 years, however, its importance has been decreasing because the therapeutic doses used are weak and little selective.

Caffeine belongs to the group of methylxantins, known for their inhibitory effect on the phosphodiesterase of cyclic nucleotides, especially AMPc, preventing its metabolism. The prolonged use of caffeine is related to uneasiness, nervousness, sleeplessness, tremors, concentration problems, heart and gastrointestinal tract disorders, as well as panic and depression syndromes. Thus, caffeine is little used in the production of pharmaceuticals [Daly J W. J. Auton. Nerv. Syst. (2000) 81, 44-52].

Some papers and patents relating to phosphodiesterase inhibitors with the use of flavonoids were found in the prior art. However, the use of dioclein and floranol and analogs, and their oral formulations using cyclodextrin has not been found.

U.S. Patent 20020132845, Guy Michael Miller; 2002 discloses compositions and methods to prevent or alleviate symptoms of ischemia of the tissues in mammals, especially of the brain tissues, using flavonoids for this purpose. However, the use of dioclein, floranol and analogs, as well as their pharmaceutical compositions is not disclosed.

Analising the patents found in the prior art, one can see that none of them uses the flavonoids described herein included in cyclodextrins, and their pharmaceutical compositions for oral use, preferably but not limited thereto, as well as their use as inhibitors of the phosphodiesterases of types 1 to 5.

The present invention is also characterized by proposing, for example, non-limiting dioclein and floranol molecules, as models for use in the study of the mechanisms of diseases such as arterial hypertension, atherosclerosis and restenosis, as well as the development of novel pharmaceutical for inhibiting phosphodiesterase 1 to 5, but preferably phosphodiesterase 1, PDE1. Thus, pharmaceuticals and their pharmaceutical compositions that inhibit PD1 are of great interest for the pharmaceutical industry, since they have a therapeutic potential for the treatment of the diseases that imply participation thereof.

Both flavonoids used in the present invention exhibit low solubility in water, instability and low or no activity when applied in oral form. So, one of the characteristics of the present technology is the increase of the solubility, stability and activity via oral route when included in cyclodextrins and when microencapsulated in biodegradable polymers.

A pharmaceutical may be chemically modified to alter its properties such as biodistribution, pharmacokinetics and solubility. A number of methods have been used to increase the solubility and stability of the drugs, among which the use of organic solvents, emulsions, liposomes, pH adjustment, chemical modifications and complexation of the pharmaceuticals with a suitable encapsulating agent such as cyclodextrins. The cyclodextrins are of the family of the cyclic oligpsaccharides that include six, seven or eight units of glucopiranose. Due to the steric interactions, the cyclodextrins form a cyclic structure in the form of a truncated cone with a non-polar internal cavity. These are chemically stable compounds that may be modified in a regioselective manner.

The cyclodextrins (hosts) form complexes with various hydrophobic molecules (guests), including them in a complete manner or in part in the cavity. The cyclodextrins have been used for solubilization and encapsulation of drugs, perfumes and flavorings, as described by Szejtli [Szejtli J., Chem. Rev., (1998) 98, 1743-1753; Szejtli J., J. Mater. Chem. (1997) 7, 575-587]. According to detailed studies of toxicity, mutagenicity, teratogenicity and carcinogenicity on cyclodextrins [Rajewski R. A. and Stella V., J. Phar. Sci., (1996) 85, 1142-1169], these have low toxicity, especially the hydroxypropyl-p-cyclodextrins, as reported by Szejtli [Szejtli J., Drug Investig., (1990) 2, 11-21]. Except for high concentrations of some derivatives, which cause damage to the erithrocytes, these products generally do not entail risk to health.

The use of the cyclodextrins as additives in foods has already been authorized in countries such as Japan and Hungary, and for more specific applications, in France and Denmark. In addition, they are obtained from a renewable source from degradation of starch. All these characteristics are a growing motivation for the discovery of new applications. The structure of the cyclodextrine molecule is similar to that of a truncated cone, low symmetry, approximately Cn. The primary hydroxyls are located on the narrower side of the cone and the secondary hydroxyls are located on the wider side. In spite of the stability conferred to the cone by the intramolecular hydrogen bonds, the latter is flexible enough to enable a considerable deviation from the regular form.

The cyclodextrins are moderately soluble in water, methanol and ethanol and readily soluble in aprotic polar solvents, such as dimethyl sulfoxide, dimethylformamide, N, N-dimethylacetamide and pyridine.

There are numberless papers in the literature on the effects of increasing the solubility in water of guests that are little soluble in water, using the ciclodextrins via inclusion compounds, as well as a discussion of the stability of the inclusion complexes, these physical-chemical characteristics have been described [Szejtli J., Chem, Rev., (1998) 98, 1743-1753; Szejtli J., J. Mater. Chem, (1997) 7, 575-587].

In addition to the cyclodextrins, biodegradable polymers are also used, which decrease the velocity of absorption of pharmaceuticals in the organism, through the controlled-release devices. In these systems the drugs are incorporated in a polymeric matrix based on the encapsulation of drugs in microspheres, which release the drug inside the organism, in small and controllable daily doses, for days, months or even years.

A number of polymers have been tested in controlled-release systems. Many have been tested due to their physical properties such as: poly (urethanes) for their elasticity, poly (siloxanes) or silicone because they are good insulators, poly (methylmetacrylate) for its physical strength, poly (vinyl alcohol) for its hydrophobicity and resistance, poly (ethylene) for its hardness and impermeability [Gilding, D. K. Biodeg. Polym. Biocompat. Clin Implat. Mater. (1981) 2, 209-232].

However, for use on humans, the material must be chemically inert and free from impurities. Some of the materials used in release systems are: poly(2-hydroxy-ethylmetacrilate), polyacrylamide, polymers based on lactic acid (PLA), based on glycolic acid (PGA), and the respective co-polymers (pLGA) and the poly(anhydrous) such as polymers based on sebasic acid (PSA) and the co-polymers with more hydrophobic polymers.

The development of new pharmaceutical formulations tends to alter the present concept of medicament. So, in the last few years a number of systems have been developed for administering pharmaceuticals to moderate the kinetics of release, improve the absorption, increase the stability of the pharmaceutical or vectored to a determined cellular population. Thus, the polymeric compositions, cyclodextrins, liposomes, emulsions, multiple emulsions have arisen, which serve as carriers for the active principles. These formulations may be administered via intramuscular injection intravenous, subcutaneous injection, oral formulation, inhalation or as devices that may be implanted or injected.

The inclusion compounds of dioclein, non-limiting example the cyclodextrins, were characterized by the physico-chemical techniques of analyses like spectroscopy of absorption in the infrared region, IR, thermal analysis (TG/DTG) and X-ray diffractions and nuclear magnetic resonance of ¹H and ¹³C.

The inhibitory activity of dioclein and of floranol, as well as that of the inclusion compounds with cyclodextrins, can be better understood from the following description:

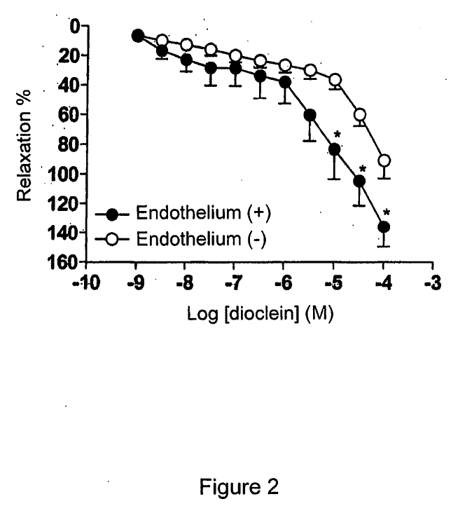

FIG. 2 represent the vasodilating effect of dioclein in the human saphenous vein, pre-contracted with phenylephrine (3×10⁻⁶M) in the presence or absence of functional endothelium. The relaxation data represent the percentage of reduction of the contraction by phenylephrine in response to dioclein and have been expressed on average±SEM. *P<0.05 (two-way ANOVA with post-test comparison BONFERRONI. The vessels of 8 patients with and 8 without functional endothelium were analyzed.

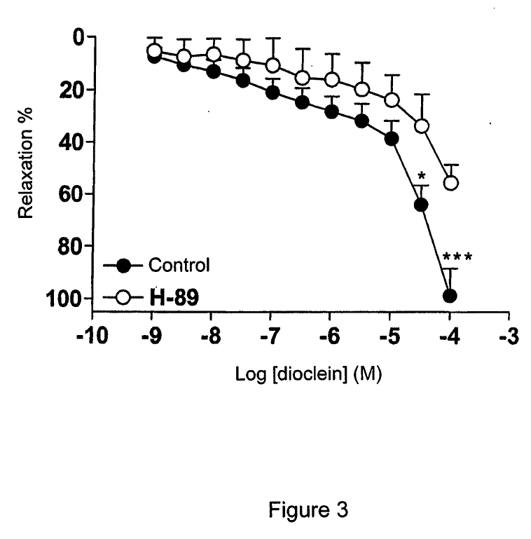

FIG. 3 shows the effect of H-89 (1 μM) on the relaxation induced by dioclein on the human saphenous vein without functional endothelium, pre-contracted with phenylephrine (3×10⁻⁶M). The data represent the percentage of reduction of the contraction by phenylephrine in response to dioclein and have been expressed average±SEM. *P<0.05. ***P<0.001 (two-way ANOVA with post-test comparison BONFERRONI). One has analyzed 8 vessels of the control group, 5 vessels of the group incubated with H-89.

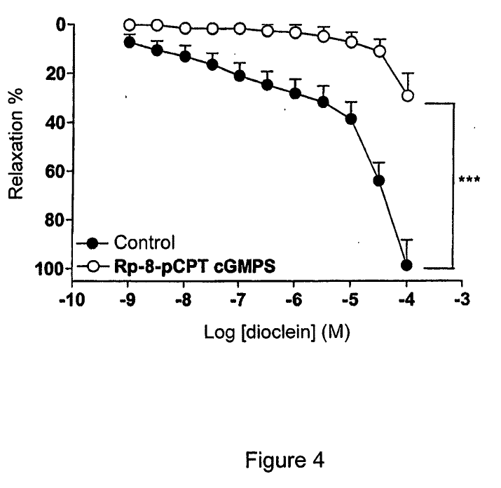

FIG. 4 illustrates the effect of Rp-8-pCPT cGMPS (10 μM) on the relaxation induced by dioclein on the human saphenous vein without functional endothelium, pre-contracted with phenylephrine (3×10⁻⁶M). The data represent the percentage of reduction of the contraction with phenylephrine in response to dioclein and have been expressed in average±SEM. ***P<0.001 (two-way ANOVA with post-test comparison of BONFERRONI). One has analyzed 8 vessels of the control group and 5 vessels of the group incubated with Rp-8-pCPT cGMPS.

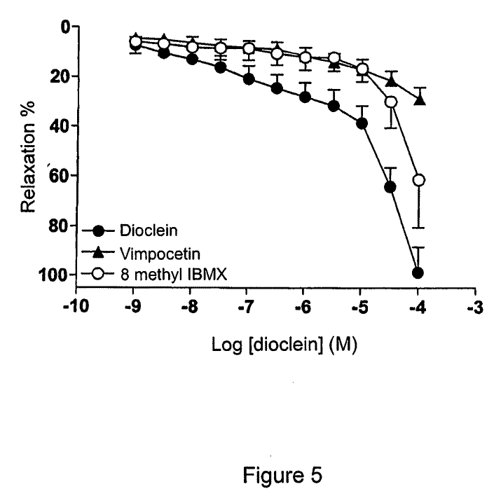

FIG. 5 shows the vasodilating effect of dioclein in comparison with that of vinpocetine and that of 8-MM-IBMX on the human saphenous vein without functional endothelium, pre-contracted with phenylephrine (3×10⁻⁶M). The data represent the percentage of reduction of the contraction with phenylephrine in response to dioclein and have been expressed in average±SEM. One has analyzed 8 vessels of the dioclein group, 7 vessels of the 8-MM-IBMX group and 9 vessels of the vinpocetine group.

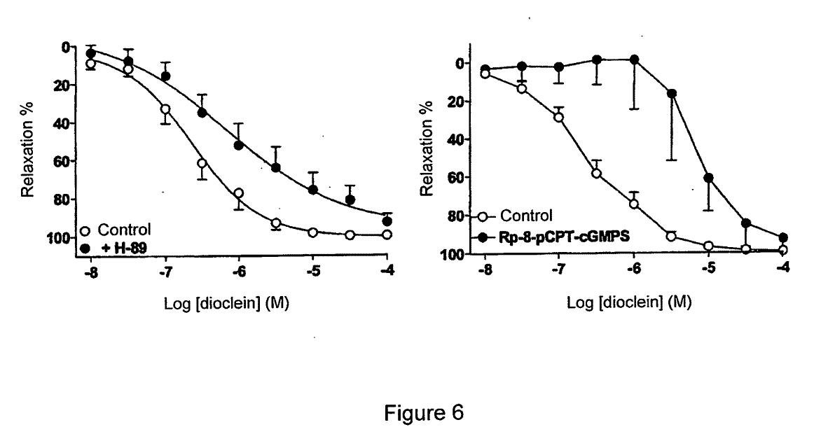

FIG. 6 is a graph that evidences the effect of H-89 (1 μM) (a) and of Rp-8-pCPT cGMPS (3 μM) (b) on the relaxation induced by dioclein in the mesenteric artery of rat, pre-contracted with phenylephrine (3×10⁻⁶M). The data represent the percentage of reduction of the contraction of phenylephrine in response to dioclein and have been expressed in average±SEM. (two-way ANOVA with post-test comparison of BONFERRONI). One has analyzed 7 vessels from the control group, 7 vessels of the group incubated with H-89 and 5 incubated with Rp-8-pCPT cGMPS.

The best results of inhibition of PDE1 are represented in the table I below. The physiologic role of PDE1 is still little known. The great problem for a better understanding of its physiological role and of the therapeutic potentialities of its inhibition is the absence of specific inhibitors on the market. Two PDE1 inhibitors are presently available on the market: Vinpocetine and 8-methoxymethyl-IBMX (8-MM-IBMX). Vinpocetine shows the inhibitory effect at concentrations higher than 30 μM on PDE1 of bovine tissue (Yu J. et al., Cell. Signal., (1997) 9, 519-29] and also, at the same concentrations, inhibits PDE7 [Sasaki et al., 2000]. Further, vinpocetine is capable of directly activating potassium channels of the type sensitive to high-conductance calcium [Wu S. N. et al., Biochem. Pharmacol., (2001) 61, 877-92]. The 8-MM-IBMX (IC₅₀=8 μM) has a poor selectivity by PDE1, since it also inhibits PDE₅ with an IC₅₀ of 10 μM [Ahn H. S. et al., J. Med. Chem., (1997) 40, 2196-210]. Dioclein has a IC₅₀ of 1.4 μM, being about 30 times more potent than vinpocetine and 8 times more potent than 8-MM-IBMX.

Dioclein is also more selective, since in inhibits PDE1 at concentrations of from 20 to 100 times smaller than the concentration necessary to inhibit PDE2, PDE3, PDE4 and PDE5. Therefore, dioclein is more selective and potent than the PDE1 inhibitors presently available on the market. Thus, the development of new substances with selective PDE1 inhibitory property will contribute to the understanding of the physiological role of the PDE1 and of the therapeutic potentialities of the inhibition of this isoform of PDE. At present, vinpocetine has been clinically tested on urinary incontinency problems and acute ischemia caused by a stroke.

In addition, due to the participation of the PDEs in some known physiological phenomena, the PDE1 inhibitors have a potential of therapeutic application to cardiovascular diseases that involve proliferative inflammatory processes like restenosis, atherosclerosis and arterial hypertension. It also has a potential therapeutic use to increase the cerebral circulation and to limit tolerance to nitrates. The calmodulin inhibitors also inhibit the activity of PDEs1. However, its poor selectivity for PDEs has limited its use.

Notwithstanding, the results of the present invention are not limited to the inhibition of the isoform of PDEI; they also indicate the possibility of inhibiting the posphodiesterases of types 2 to 5, with the use of these flavonoids, but with a somewhat higher concentration.

Also, the present invention is characterized by preparing sustained as well as controlled release devices of dioclein, floranol and analogs using the cyclodextrins and the biodegradable polymers aiming at the study/inhibition of the actuation of the phosphodiesterases of types 1, 2, 3,4 and 5.

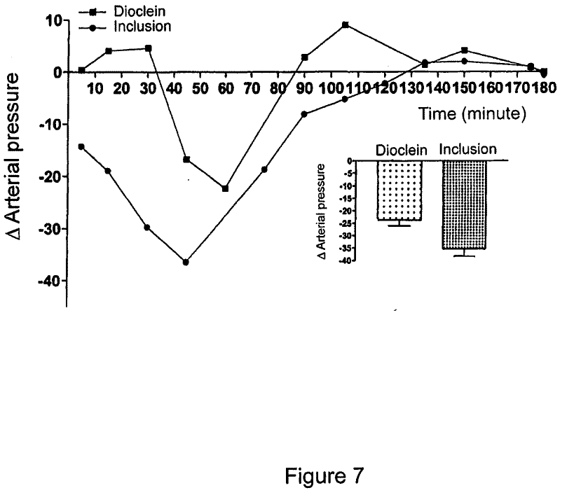

FIG. 7 is a representative example of the effect of dioclein (2.5 mg/kg) and of the inclusion product of dioclein in cyclodextrin (inclusion: 2.5 mg/kg), applied by intraperitoneal route, on the arterial pressure of mice. In the highlight we can see the average±SEM of the maximum effect achieved on 6 different mice. In these experiments dioclein and the inclusion product of dioclein in the cyclodextrin were dissolved with the aid of DMSO.

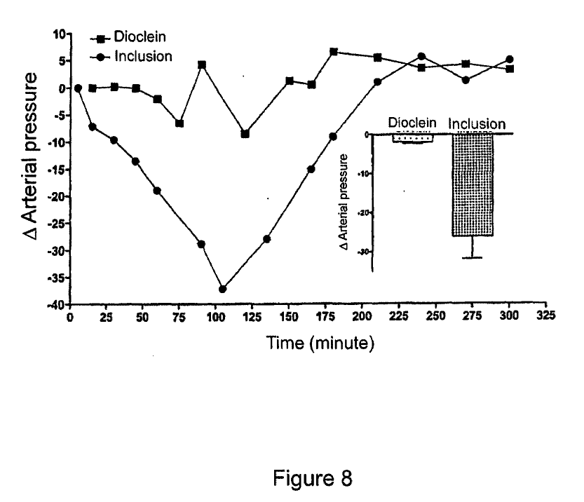

FIG. 8 is a representative example of the effect of dioclein (10 mg/kg) and of the inclusion product of dioclein in cyclodextrin (inclusion: 10 mg/kg), applied by oral route, on the arterial pressure of mice. In the highlight one can see the average±SEM of maximum effect achieve in 3 different mice. In this experiments dioclein and the inclusion product of dioclein in cyclodextrin were dissolved with the aid of DMSO.

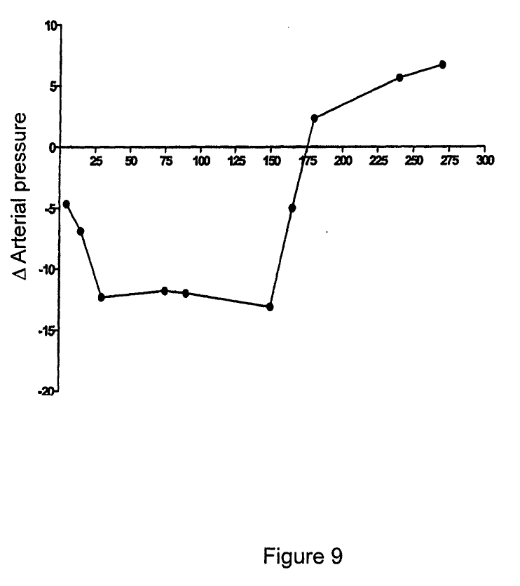

FIG. 9 is a representative example of the effect of the inclusion product of dioclein in cyclodextrin (inclusion; 10 mg/kg), solubilized in water, applied by oral route, on the arterial pressure of mice. Dioclein cannot be tested due to its very low solubility in water.

FIGS. 8 and 9 show clearly that the substances of the present invention are not active when used by oral route. The substances of the present invention are not water-soluble either. Thus, the inclusion of dioclein in the cyclodextrins has enabled its solubility in water and an activity by oral route.

The present invention will be better understood with the help of the following non-limiting examples.

### EXAMPLE 1

### Evaluation of the PDEs Inhibiting Effect of the Flavonoids Included or Not in Cyclodextrins as a Non-Limiting Example

The substances developed in the present invention have been tested for their ability of inhibiting different isoforms of PDEs.

Table 1 shows the inhibitory effect of dioclein and of floranol, molecules of the present invention on PDE1, PDE3, PDE4 and PDE5 isolated from the smooth muscle of ox aorta and on the PDE2 isolated from human platelets.

TABLE I
Values of IC₅₀ of dioclein and of floranol on the various isoforms
of phosphodiesterases existing in the vascular smooth musculature.

| Different isoforms | Dioclein (μM) | Floranol μM) |
|---|---|---|
| PDE1 − calmodulin | 2.47 ± 0.26; Ki = 0.62 | 2.75 ± 0.20 |
| PDE1 + calmodulin | 1.44 ± 0.35, Ki = 0.59 | 3.06 ± 0.14 |
| Basal PDE2 | 100.0 ± 0.50 | 26.8 ± 4.62 |
| Activated PDE2 (+GMPc) | 38.15 ± 8.92 | 65.3 ± 6.87 |
| PDE3 | 28.07 ± 0.43 | 47.07 ± 5.25 |
| PDE4 | 16.78 ± 1.42 | 11.0 ± 2.31 |
| PDE5 | 23.0 ± 5.50 | 7.14 ± 39 |

One observes that dioclein and floranol are potent and selective PDE1 inhibitors. The compounds of the present invention are more effective with regard to potency and selectivity than the other two single PDE1 inhibitors presently available on the market: Vinpocetine and 8-methoxymethyl-IBMX (8-MM-IBMX). Vinpocetine shows an inhibitory effect at concentrations higher than 30 μM in PDE1 of bovine tissue [Yu J. et al., Cell. Signal., (1997) 9, 519-29] and also, at the same concentrations, inhibits PDE7 [Sasaki et al., 2000]. Further, vinpocetine is capable of directly activating potassium channels of the type sensitive to high-conductance calcium [Wu S. N. et al., Biochem. Pharmacol., (2001) 61, 877-92]. 8-MM-IBMX (IC₅₀=8 μM) has a poor selectivity for PDE1, since it also inhibits PDE5 with an IC₅₀ of 10 μM [Ahn H. S. et al., J. Med. Chem., (1997) 40, 2196-210]. Dioclein has a Cl₅₀ of 1.4 μM, being about 30 times more potent than vinpocetine and 8 times more potent than 8-MM-IBMX.

Dioclein is also more selective, since it inhibits PDEI at concentrations of 20-100 times smaller than the necessary to inhibit PDE2, PDE3, PDE4 and PDE5. Therefore, dioclein is more selective and more potent than the PDE1 inhibitors presently available on the market.

### EXAMPLE 2

### Evaluation of the Vasodilating Effect of Dioclein Dependent Upon the Inhibition of PDEs, as a Non-Limiting Example

FIG. 2 illustrates the effect of the flavonoids of the present invention on the human saphenous vein. This graph shows the vasodilating effect of dioclein in the presence (Cl₅₀=3.0±0.2 μM) and in the absence (Cl₅₀=11±0.4 μM) of functional endothelium. FIG. 3 illustrates the effect of dioclein on the human saphenous vein without functional endothelium, in the absence and in the presence of an inhibitor selective of protein Kinase A, which is the intracellular receptor of AMPc. The vasodilating effect of dioclein was displaced to the right in the presence of H-89 (inhibitor of the protein Kinase A), showing that the AMPc is involved in its vasodilating effect.

FIG. 4 shows that the vasodilating effect of the flavonoids of the present invention on the human saphenous vein was almost totally blocked in the presence of an inhibitor selective of the protein kinase G (Rp-8-pCPT-cGMPS). The protein Kinase G is the intracellular receptor of GMPC. The results of FIGS. 3 and 4 show that the vasodilating effect of dioclein on the human saphenous vein is mediated by an intracellular increase of the cyclic nucleotides. These results together with those of Table 1 show that the vasodilating effect of dioclein on the human saphenous vein is due to an inhibition of PDEs. The Cl₅₀ of the vasodilating effect of dioclein on the human saphenous vein of 3.0±0.2 μM correlate well with the Cl₅₀ 1.44±0.35 μM of its inhibitory effect on the PDE1. The fact that the vasodilating effect of dioclein is mediated by the GMPc and by the AMPc also correlates well with the characteristics of the PDE1 that hydrolyzes the two types of cyclic nucleotides. In the human saphenous vein, one of the PDEs described is the PDE1 [Wallis R. M. et al., Am. J. Cardiol., (1999) 83, 3C-12C), which is also the isoform related to the processes of stenosis and obstruction of the vein after manipulation [Ryabaklin S. D. et al., J. Clin. Invest., (1997) 100, 2611-16211.

FIG. 5 compares the vasodilating effect of dioclein with that of Vinpocetine and of 8-MM-IBMX on the human saphenous vein. We can note that dioclein is much more potent than the two conventional PDE1 inhibitors. Dioclein causes the human saphenous vein to relax (in the absence of functional endothelium) with a Cl₅₀ of 11.1±2.7 μM, whereas 8-MM-IBMx had a Cl₅₀ of 30.9±16.0 μM. Vinpocetine produced only 30% of maximum effect.

FIG. 6 shows that the vasodilating effect of the flavonoids of the present invention on the mesenteric artery of rat also decreases in the presence of H-89 (a) and Rp-8-pCPT-cGMPS (b) and, therefore, mediated by the cyclic nucleotides AMPc and GMPc.

### EXAMPLE 3

### Preparation of the Inclusion Compounds 1:1 of Dioclein with β-Cyclodextrin

The dioclein, DC, used (MM_{DC}=332.31 g/mol) was synthesized according to the technique described by Spearing P. et al. [J. Nat. Prod., (1997) 60, 399-400] and β-cyclodextrin (β-CD): MM_{β-CD}=1,135.01 g/mol, from Aldrich Chemical Compay, Inc. USA.

One weighed 102.5 mg of β-CD, which was dissolved with 5 ml of distilled water (with a slight warming, maximum 50° C.) in a beaker. After the spontaneous cooling, one added 30.0 mg of DC, stirring (in a magnetic stirrer) for about 2 hours. The beaker was protected from luminosity (pharmaceutical easy to decompose and oxidize). The compound was lyophilized for 48 hs, after being frozen in nitrogen, and characterized by physico-chemical techniques of analysis.

The absorption spectra in the infrared region were recorded on the spectrophotometer IRTF Galaxy 3000 Mattson in the range of 4000-400 cm⁻¹, using KBr tablets. The TG/DTG curves were obtained on TGA-50H thermo balance from Shimadzu, under a dynamic N₂ atmosphere with flow rate of approximately 100 mL/min, using alumina melting pot and a heating rate of 10° C./min. The samples were heated from 25 to 750° C. The DSC curves using the DSC-50 system of Shimadzu, under a dynamic N₂ atmosphere with flow rate of 50 mL/min, alumina melting pot, heating rate of 10° C./min. The X-ray diffractgrams were recorded on the apparatus Rigaku Geiger-flex 2037, using Cu tube and radiation Cu Kα=1.54051, angles of 2θ ranging from 2 to 600. The NMR spectra were recorded, by using the spectrophotometer Bruker DPX-200 (200 MHz), using DMSO or D₂O as a solvent and TMS as an internal standard.

To characterize the DC, one used the absorption spectroscopy techniques in the infrared region (IR), thermal analysis (TGA/DTG), X-ray diffraction and nuclear magnetic resonance (NMR) of ¹H and ¹³C.

The main characteristic bands are presented in Table II, wherein the attributions were made with the aid of the literature (Silvertein, R. M., Wegster, F. X., Identificação Espectrométrica de Compostos Orgânicos, 6^{th} ed. Livros Técnicos e Cientificos Editora S. A , 2000).

Examining this table one can identify the main functional groups occurring in the DC molecule.

Examining the TGA and DTG curves for DC one can initially see a level of thermal stability in the temperature range of 25-200° C. Subsequently, one observes an intense process of thermo decomposition in the temperature range of 240-700° C., which corresponds to 71% of the loss of mass. It is important to point out that the residue obtained was quite marked, this being an organic compound. At present, one is carrying out physico-chemical analyses in order to know the nature of this residue better.

TABLE II
Main absorption bands in the IR for the DC

| Signal (cm⁻¹) | Attribution (attempt) |
|---|---|
| 3,500 | νOH |
| 3,200 | νOH |
| 3,050 | νC—H |
| 1,660 | νC═O(ketone) |
| 1,500 | νC═C |
| 1,310 | δOH (phenol) |
| 1.200 | νC—H (phenol) |
| 800 | δC—H (arom.) |

The X-ray diffractgram of the DC of 4 to 60° 2θ suggests a semi crystalline structure thereof, showing marked peaks and an amorphousness halo, between 15 and 40 ° 2θ.

The data of the spectra of NMR of ¹H and of ¹³C of the dioclein, achieved in DMSO, are represented in Table III and IV below.

TABLE III
Chemical displacements and relaxation times
of NMR of ¹H of the DC in DMSO (400 MHz)

| | | |
|---|---|---|
| Hydrogen | δ(ppm) | T1 (s) |

| | | | |
|---|---|---|---|
| 2 (OCH₃)* | 3.66 | (s) | 0.956 |
| 3 (OCH₃)* | 3.85 | (s) | 0.658 |
| 1 (OH) | 11.92 | (sd) | 1.892 |
| H₄ | 6.29 | (s) | 1.369 |
| H₈₍ₐₓᵢₐₗ₎ | 2.75 | (dd) | 0.381 |
| H_{8(eq)} | 3.16 | (dd) | 0.366 |
| H₉ | 5.67 | (dd) | 1.502 |
| H_{2′} | 6.87 | (d) | 1.465 |
| H_{4′} | 6.61 | (dd) | 1.639 |
| H_{5′} | 6.69 | (d) | 1.153 |
| 3′ (OH) | 8.81 | (sd) | 1.596 |
| 6′ (OH) | 9.07 | (sd) | 1.612 |

*Confirmed by NOE

TABLE IV
Chemical displacements and relaxation
times of NMR of ¹³C of the DC in DMSO

| Carbon | δ(ppm) |
|---|---|
| C₁ (—C) | 154.254 |
| C₂ (—C) | 129.678 |
| C₃ (—C) | 160.707 |
| C₄ (CH) | 91.992 |
| C₅ (—C) | 158.958 |
| C₆ (—C) | 102.567 |
| C₇ (C═O) | 197.491 |
| C₈ (CH₂) | 41.27 |
| C₉ (CH) | 74.293 |
| 2 (OCH₃) | 60.053 |
| 3 (OCH₃) | 56.297 |
| C_{1′}(—C) | 146.446 |
| C_{2′}(CH) | 113.179 |
| C_{3′}(—C) | 150.061 |
| C_{4′}(CH) | 115.811 |
| C_{5′}(CH) | 116.239 |
| C_{6′}(—C) | 125.285 |

The results of the analyses of NMR for the DC were compatible with the literature (Silvertein, R. M., Webster, F. X., Identificação Espectrométrica de Compostos Orgânicos, 6^{a}^{^{a}} ed, Livros Técnicos e Cientificos Editora S. A. 2000).

### EXAMPLE 4

### Physico-Chemical Characterization of the Inclusion Compound

To characterize the inclusion compound (IC), one used the techniques of absorption spectroscopy in the IR region, thermal analysis (TG and DTG), X-ray diffraction in powder and NMR of ¹H.

Examining the absorption spectra in the IR region of the DC, of the β-CD and IC and MM (mechanical mixture of β-CD and DC), one can observe: the more characteristic absorptions of the DC have already been discussed. For β-CD, the spectrum presented a broad band around 3.500 cm⁻¹ attributed to the stretching of the various O—H bonds, many of them involved in hydrogen bonds. One can also observe bands at 2.910 cm⁻¹ referring to the ν_{C-H} at 1.640 cm⁻¹ corresponding to δ_{OH} and at 1.100 cm⁻¹ corresponding to the vibration frequency of the C—O—C groups [Szejti J., Chem., Rev., (1998) 98,1743-1753].

Comparing the spectra of the IC with that of the β-CD, one observes that some bands characteristic of the β-CD, stretching OH and C—H, deformation OH and stretching C—O—C appear again in the spectrum of the IC without chemical displacement. However, one can observe minor modifications like the tapering of the ν_{OH} at 3,500 cm⁻¹ and alterations of the bands of ν_{C-O-C} around 1,100 cm⁻¹. In contrast, comparing the spectra of IC and of free DC, one observes major alterations in the bands of ν_{OH} at 3,500 cm⁻¹ and in the bands referring to the stretchings C═C, deformations C—H and OH of dioclein aromatics in the range of 1,600-800 cm⁻¹. However, in comparing the spectra of MM with that of β-CD and of DC, what one basically observes is an overlapping of the two spectra (β-CD, DC). Further, one can point out the bands corresponding to the vibration frequencies of the C—O—C groups at 1,100 cm⁻¹ of β-CD and a little defined overlapping in the range of 1,600-800 cm¹, which embraces bands referring to the stretchings C═C, deformations C—H and OH of dioclein aromatics.

From the observations made, one can say that the results of this analysis of IR indicate the formation of a novel compound, since in the suggested inclusion compound the characteristic bands of DC undergo major alterations when compared with those observed for the mechanical mixture.

In the TG curve corresponding to the β-CD, initially one can see a loss of mass in the range of 25-100° C. referring to the water outlet. Then, a stability level occurs between 100 and 300° C., where the complete decomposition begins, with a maximum of loss of mass at the temperature of 330° C. The residue obtained corresponds to less than 3% of the total mass. When this termal behavior of the β-CD is compared with the TG curves of IC and of MM, one notes an increase of about 20° C., at most, in the inflection of the decline curve of IC, that is to say, increase of its thermal stability, whereas the behavior of MM is significantly similar to that of β-CD, except for the higher final residue, close to 9%. These results are indicative of the formation of a novel compound.

Analyzing the DSC's curves, one can see that, in the case of β-CD, there are three thermal events, two of them being endothermic and one exothermic at 70° C., 270-300° and 320° C., respectively, associated to the exit of water molecules, fusion with caramelization of β-CD and decomposition thereof. On the other hand, the DSC curve of dioclein has two events, one endothermic at 250° C. and the other exothermic at 270° C., the first one being associated to the fusion of DC and the second one corresponding to the thermo decomposition.

The DSC curve of the inclusion compound has a thermo decomposition profile different from the free materials and from the respective mechanical mixture, but no peak of fusion of DC at 250° C. is observed, which suggests the formation of a new crystalline phase after the interaction of DC with β-CD.

The X-ray diffractgrams of DC, β-CD, MM and IC allow one to observe that: IC has an amorphous structure due to the marked amorphousness halo observed in the range of from 15 to 40° 2θ. This halo also appears in the diffractogram of DC, but with less intensity; however, in IC it is not observed the intense peaks of crystallinity. This structure, comparatively more amorphous, suggests the formation of a novel compound, since the diffractogram of MM has the peaks of crystallinity of β-CD in addition to the amorphousness halo of DC.

TABLE V
Chemical displacements and relaxation
times of NMR or ¹H of β-CD in DMSO.

| Hydrogen | δ(ppm) | T₁(s) |
|---|---|---|
| H₁ | 4.84 (d; 3.28) | 1.049 |
| H₂ | 3.31 | 1.182 |
| H₃ | 3.64 | 0.969 |
| H₄ | 3.34 | 1.009 |
| H₅ | 3.58 | 1.036 |
| H₆(a, b) | 3.64 | 0.969 |
| OH (2) | 5.70 | 1.169 |
| OH (3) | 5.66 | 1.146 |
| OH (6) | 4.43 | 1.165 |

According to the results, one can observe that, in IC, T₁ increased to H₁ and decreased to OH (2), OH (3), OH (6) when compared with the values of β-CD alone. This indicates the modification in the intense movement of the pyranose rings as a result of the complexation. A decrease in the value of T₁ suggests the decrease of the molecular movement due to interaction with DC.

Also in the comparison of the values of T₁ for DC in IC and free DC, one observes positive and negative variations that confirm the occurrence of interaction between it and β-CD.

TABLE VI
Chemical displacements and relaxation times
(T₁) of NMR of ¹H of DC and of β-CD in IC
and the respective variations between the T₁

| ¹H of DC in IC | δ(ppm) | | T₁(s) | T1_{(CI)}-T_{1(DC)} |
|---|---|---|---|---|
| 2 (OCH₃) | 3.66 | (s) | — | — |
| 3 (OCH₃) | 3.86 | (s) | 0.93 | 0.272 |
| 1(OH) | 11.917 | (s) | 1.74 | −0.1525 |
| H₄ | 6.29 | (s) | 1.14 | −0.229 |
| H₈(ax) | 2.75 | (dd) | 0.36 | −0.021 |
| H₈(cq) | 3.16 | (dd) | 0.42 | 0.054 |
| H₉ | 5.67 | (dd) | * | 0.0 |
| H_{2′} | 6.87 | (d) | 1.35 | −0.115 |
| H_{4′} | 6.61 | (dd) | 1.53 | −0.109 |
| H_{5′} | 6.69 | (d) | 1.15 | −0.003 |
| 3′ (OH) | 8.82 | (s) | 1.47 | −0.126 |
| 6′ (OH) | 9.08 | (s) | 1.40 | −0.212 |
| 1H of β-CD in IC | δ(ppm) | | T₁(s) | T₁ (IC)-T₁ (β-CD) |
| H₁ | 4.84 | (d) | 1.16 | 0.111 |

| | | | |
|---|---|---|---|
| H₂ | 3.31 | ** | — |
| H₃ | 3.64 | ** | — |
| H₄ | 3.34 | ** | — |
| H₅ | 3.58 | ** | — |
| H₆(a,b) | 3.64 | ** | — |
| OH (2) | 5.70 | 1.09 ** | −0.079 |
| OH (3) | 5.66 | 0.99 ** | −0.156 |
| OH(6) | 4.43 | 0.70 | −0.465 |

Covered by the signal of β-CD;
** overlapped signals (error in T1);

### EXAMPLE 5

### Preparation of the Controlled Release Devices of Dioclein and of the Inclusion compounds in Cyclodextrins, Using the Microspheres of the Biodegradable Polymers PLGA, as a Non-Limiting Example

First, one prepares an emulsion constituted by an organic phase constituted by poly (lactic-glycolic acid) (PLGA) dissolved in dichloromethane and an aqueous phase constituted by the flavonoids, dioclein and floranol, as an example. This emulsion is then subjected to sonication for half a minute, and then 1% polyvinyl alcohol (PVA) solution is added, thus forming a second emulsion, which undergoes stirring for 1 minute for complete homogenization of the emulsion. The system is kept under agitation without heating for 2 hours, so that the solvent can evaporate. Then, the mixture is centrifuged 2 to 3 times, the supernatant being removed and washing with water is carried out. In the end, 1-2mL of water is left, and the system obtained is subjected to lyophilization for 24-48 hours.

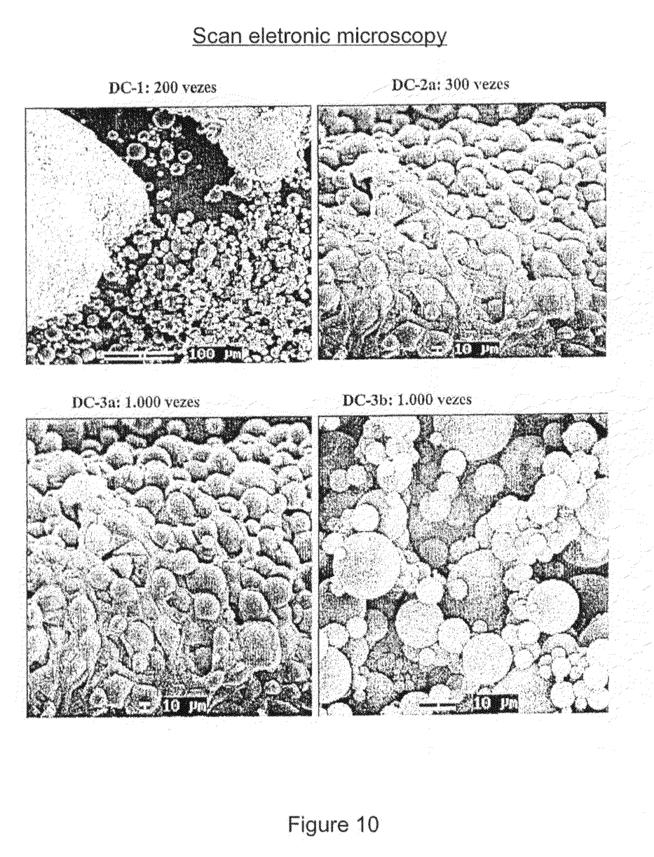

The microspheres are then characterized through the thermal analysis. The DSC curve obtained from the glass transition, exhibiting a value close to that of the polymer (PLGA). The micrographics obtained by electronic scan microscopy (SEM) enabled one to verify the average particle size of 10-30 microns, FIG. 10. One further observes the smooth surface of the microspheres. The images were obtained with a JFM 480A type electronic microscope, the samples having been covered with 99% gold for 240 seconds.

In order to determine the encapsulating capacity of the different system used, one constructed UV-VIS calibration curves, obtaining a relation between concentration and absorbance, thus being able to determine the amount of flavonoid incorporated into the microspheres of biodegradable polymer.

One carried out the tests for controlled release of DC, and its respective inclusion compound in cyclodextrins from the devices, based on biodegradable polymers.

### EXAMPLE 6

### Evaluation of the Hypotensor Effect of Flavonoids Included or not in Cyclodextrins as a Non-Limiting Example

The substances developed in the present invention have been tested for their ability of producing hypotension in animal models.

FIG. 7 illustrates the effect of dioclein and of dioclein included in cyclodextrin, dissolved with the aid of DMSO on the arterial pressure of mice. One can observe that, when administered by intraperitoneal route, both dioclein and the inclusion compound of dioclein reduced the arterial pressure of mice. However, the effect of the inclusion product was more marked and more prolonged, showing that cyclodextrin improves the bioavailability of dioclein. FIG. 8 illustrates the effect of dioclein and of dioclein included in cyclodextrin dissolved with the aid of DMSO on the arterial pressure of mice when applied by oral route (gavage). One can observe that dioclein is not active via oral route. However, the inclusion compound is active, even when administered by oral route. FIG. 9 illustrates the effect of included dioclein, dissolved in water. Dioclein without inclusion in cyclodextrin cannot be tested due to its insolubility in water. One can observe that dioclein included in cyclodextrin maintains its effect by oral route even when water is used as a carrier for dissolving it.

1-13. (canceled)

14. A pharmaceutical composition comprising substances as potent and selective inhibitors of the isoforms of phosphodiesterase of types 1 to 5 (PDE1, PDE2, PDE3, PDE4 and PDE5).

15. A pharmaceutical composition which is comprised of:
(a) a compound based on dioclein, floranol or an analog thereof having inhibitory activity on phosphodiesterase type 5 (PDE5) of general formula according to the following 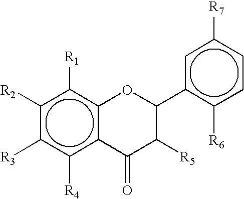
wherein R1, R2, R3, R4,R5, R6 and R7 are functional groups that are the same or different and selected from the group consisting of hydrogen, hydroxyl, methoxyl and prenyl;
(b) inclusion complexes between said compound and cyclodextrin or a derivative thereof;
(c) said compound and pharmaceutically and pharmacologically acceptable excipients; or
(d) controlled release devices of said compound or said inclusion complexes with a biodegradable polymer.

16. The composition of claim 15, wherein said biodegradable polymer is selected from the group consisting of as poly(lactic acid) (PLA), poly(glycolic acid) (PGA), poly(lactic-glycolic acid) (PLGA) and mixtures thereof.

17. The composition of 15, wherein said compound is active by oral route.

18. A process of preparing controlled release devices comprising substances as potent and selective inhibitors of isoforms of phosphodiesterases as defined in claim 14, which comprises:
(a) preparing a first emulsion comprising (i) an organic phase comprising poly(lactic-glycolic acid) (PLGA) dissolved in solvent and (ii) an aqueous phase comprising dioclein, floranol or an analog thereof;
(b) subjecting said emulsion to sonication and then adding polyvinyl alcohol (PVA) to form a second emulsion;
(c) stirring said second emulsion until homogenized;
(d) agitating said homogenized emulsion and evaporating solvent to form microspheres containing the substances in a mixture;
(e) centrifuging said mixture, removing supernatant, and washing microspheres with a solution; and
(f) lyophilizing any remaining solution to prepare said controlled release devices.

19. A method of selectively inhibiting activity and potency of a phosphodiesterase in a patient in need of such treatment, which comprises administrating the composition of claim 14 to the patient.

20. The method according to claim 19, wherein the phosphodiesterase is selected from the group consisting of phosphodiesterase type 1 (PDE1), phosphodiesterase type 2 (PDE2), phosphodiesterase type 3 (PDE3), phosphodiesterase type 4 (PDE4) and phosphodiesterase type 5 (PDE5).

21. The method according to claim 19, wherein the phosphodiesterase is PDE1.

22. The method according to claim 19, wherein the phosphodiesterase is PDE5.

23. The method according to claim 19, wherein administration is by an intramuscular, oral, intravenous, subcutaneous, topical or inhalation (pulmonary, intranasal, or intrabuccal) route or a device that can be implanted or injected.

24. A method to effect vasodilation to a patient in need of such treatment, which comprises administrating the composition of claim 14 to the patient to obtain vasodilating activity on human arteries and veins via activation of protein kinase A and protein kinase G.

25. The method according to claim 24, wherein administration is by an intramuscular, oral, intravenous, subcutaneous, topical or inhalation (pulmonary, intranasal or intrabuccal) route or a controlled release device that can be implanted or injected.

26. A method to effect vasodilation to a patient in need of such treatment, which comprises administrating the composition of claim 14 to the patient to obtain vasodilating activity on human arteries of resistance via activation of protein kinase A and protein kinase G.

27. The method according to claim 26, wherein administration is by an intramuscular, oral, intravenous, subcutaneous, topical or inhalation (pulmonary, intranasal or intrabuccal) route or a controlled release device that can be implanted or injected.

28. A method of treating at least arterial hypertension, atherosclerosis or restenosis in a patient in need of such treatment, which comprises administrating the composition of claim 14 to the patient to treat at least arterial hypertension, atherosclerosis or restenosis.

29. The method according to claim 28, wherein administration is by an intramuscular, oral, intravenous, subcutaneous, topical or inhalation (pulmonary, intranasal or intrabuccal) route or a controlled release device that can be implanted or injected.

30. A method to increase bioavailability of dioclein, floranol or an analog thereof, which comprises administrating the composition of claim 14 by an intramuscular, oral, intravenous, subcutaneous, topical, inhalation (pulmonary, intranasal or intrabuccal) route or as a controlled release device that can be implanted or injected to increase bioavailability of the compound.

31. A process to obtain molecular models for development of pharmaceutical compounds and compositions based on dioclein, floranol, or an analog thereof, which comprises using the composition of claim 14.
